(19) United States
(12) Patent Application Publication
(10) Pub. No.: US 2004/0241105 A1
(43) Pub. Date: Dec. 2, 2004

(54) Self-foaming or foamy preparations containing inorganic gel formers, organic hydrocolloids and particulate hydrophobic, hydrophobicized or oil-absorbing solid-body substances

(75) Inventors: Hiedi Riedel, Hamburg (DE); Andreas Bleckmann, Hamburg (DE); Rainer Kropke, Schenefeld (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/796,850

(22) Filed: Mar. 8, 2004

Related U.S. Application Data
(63) Continuation of application No. PCT/EP02/09981, filed on Sep. 6, 2002.

(30) Foreign Application Priority Data
Sep. 7, 2001 (DE) 101 44 061.8

(51) Int. Cl. A61K 9/00; A61K 7/00

(52) U.S. Cl. 424/47

(57) ABSTRACT

The invention a self-foaming or foamy cosmetic or dermatological preparation comprising a particular emulsifier system, at least one gas, one or more gel formers, one or more organic hydrocolloids, and one or more particulate hydrophobic, hydrophobicized or oil-absorbing solid-body substances. The present invention also includes a method of applying the preparation to the skin or hair.

### CROSS-REFERENCE TO RELATED APPLICATIONS

This is a continuation application of PCT/EP02/09981, filed Sep. 6, 2002, which is incorporated herein by reference in its entirety, and also claims the benefit of German Priority Application No. 101 44 061.8, filed Sep. 7, 2001.

### FIELD OF THE INVENTION

The present invention relates to self-foaming or foamy cosmetic or dermatological preparations, and particularly skincare cosmetic or dermatological preparations.

### BACKGROUND OF THE INVENTION

Foams or foamy preparations are a type of disperse system.

By far the most important and best known disperse system are emulsions. Emulsions are two- or multi-phase systems of two or more liquids which are insoluble or only slightly soluble in one another. The liquids (pure or as solutions) are present in an emulsion in a more or less fine distribution, which generally has only limited stability.

Foams are structures of gas-filled, spherical or polyhedral cells which are delimited by liquid, semiliquid, high-viscosity or solid cell ribs. The cell ribs, connected via points of intersection, form a continuous framework. The foam lamellae stretch between the cell ribs (closed-cell foam). If the foam lamellae are disturbed or if they flow back into the cell rib at the end of foam formation, an open-cell foam is obtained. Foams are also thermodynamically unstable since a reduction in the surface area leads to the production of surface energy. The stability and thus the existence of a foam is thus dependent on to what extent it is possible to prevent its self-destruction.

Cosmetic foams are usually dispersed systems of liquids and gases, where the liquid represents the dispersant and the gas represents the dispersed substance. Foams of low-viscosity liquids are temporarily stabilized by surface-active substances (surfactants, foam stabilizers). Because of their large internal surface area, such surfactant foams have a high adsorption capacity, which is utilized, for example, in cleaning and washing operations. Accordingly, cosmetic foams are used, in particular, in the fields of cleansing, for example as shaving foam, and of haircare.

To generate foam, gas is bubbled into suitable liquids, or foam formation is achieved by vigorously beating, shaking, spraying or stirring the liquid in the gas atmosphere in question, provided that the liquids comprise suitable surfactants or other interface-active substances (“foam formers”), which, apart from interfacial activity, also have a certain film-forming ability.

Cosmetic foams have the advantage over other cosmetic preparations of permitting a fine distribution of active ingredients on the skin. However, cosmetic foams can generally only be achieved using particular surfactants, which, moreover, are often not well tolerated by the skin.

A further disadvantage of the prior art is that such foams have only low stability, for which reason they usually collapse within approximately 24 hours. A requirement of cosmetic preparations, however, is that they have stability for years, as far as possible. This problem is generally taken into account by the fact that the consumer produces the actual foam himself just before use using a suitable spray system, for which purpose, for example, it is possible to use spray cans in which a liquefied pressurized gas serves as propellant gas. Upon opening the pressure valve, the propellant liquid mixture escapes through a fine nozzle, and the propellant evaporates, leaving behind a foam.

After-foaming cosmetic preparations are also known per se. They are firstly applied to the skin from an aerosol container in flowable form and, after a short delay, develop the actual foam only once they are on the skin under the effect of the after-foaming agent present, for example a shaving foam. After-foaming preparations are often in specific formulation forms, such as, for example, after-foaming shaving gels or the like.

However, the prior art does not include any sort of cosmetic or dermatological preparations which could be foamed as early as during the preparation and nevertheless have a sufficiently high stability in order to be packaged in the usual manner, stored and put onto the market.

### SUMMARY OF THE INVENTION

An object of the present invention was therefore to enrich the prior art and to provide cosmetic or dermatological self-foaming or foamy preparations which do not have the disadvantages of the prior art.

German laid-open specification DE 197 54 659 discloses that carbon dioxide is a suitable active ingredient for stabilizing or increasing the epidermal ceramide synthesis rate, which may serve to enhance the permeability barrier, reduce the transepidermal water loss and increase the relative skin moisture. To treat the skin, the CO₂ is, for example, dissolved in water, which is then used to rinse the skin. However, the prior art hitherto does not include any sort of cosmetic or dermatological bases in which a gaseous active ingredient could be incorporated in an adequate, i.e. effective, concentration.

It was thus a further object of the present invention to find cosmetic or dermatological bases into which effective amounts of gaseous active ingredients can be incorporated.

It was surprising and could not have been foreseen by the person skilled in the art that self-foaming or foamy cosmetic or dermatological preparations which comprise

I. an emulsifier system which consists of

A. at least one emulsifier A chosen from the group of completely neutralized, partially neutralized or unneutralized branched or unbranched, saturated or unsaturated fatty acids with a chain length of from 10 to 40 carbon atoms,

B. at least one emulsifier B chosen from the group of polyethoxylated fatty acid esters with a chain length of from 10 to 40 carbon atoms and with a degree of ethoxylation of from 5 to 100 and

C. at least one coemulsifier C chosen from the group of saturated or unsaturated, branched or unbranched fatty alcohols with a chain length of from 10 to 40 carbon atoms,

II. up to 30% by weight—based on the total weight of the preparation—of a lipid phase,

III. 1 to 90% by volume, based on the total volume of the preparation, of at least one gas chosen from the group consisting of air, oxygen, nitrogen, helium, argon, nitrous oxide (N₂O) and carbon dioxide (CO₂),

IV. 0.01 to 10% by weight of one or more gel formers chosen from the group of inorganic thickeners,

V. one or more substances chosen from the group of organic hydrocolloids, and

VI. 0.01 to 10% by weight of one or more particulate hydrophobic or hydrophobicized or oil-absorbing solid-body substances overcome the disadvantages of the prior art.

### DETAILED DESCRIPTION OF THE PREFERRED EMBODIMENTS

According to the prior art, foamy cosmetic emulsions which are characterized by a high gas content cannot be formulated or prepared industrially without propellant gas. In particular, systems which are based on classic emulsifiers and gel formers and develop, as a result of sheering (stirring, homogenization), a foam with a high stability could only be prepared by the prior art with the help of after-foaming agents.

As a result of the invention, the introduction of gases is aided, and a stabilizing and significantly after-foaming effect is achieved over a prolonged storage period, even at relatively high temperatures (e.g. 40° C.). It was particularly surprising here that customary after-foaming agents such as, for example, propellant gases can be dispensed with. The introduction of gases is, surprisingly, extraordinarily increased compared with the prior art. Thus, for example, foam boosting with up to 100% increased gas volume can be achieved without using foaming agents customary according to the prior art, such as surfactants.

As a result of this, it is possible for the first time to stably generate formulations with an excellent, novel type of cosmetic activity and with an extraordinarily high gas volume (air and/or other gases such as oxygen, carbon dioxide, nitrogen, helium, argon etc.) over a long storage period at high temperatures. At the same time, the preparations according to the invention are characterized by above-average skincare and very good sensory properties.

For the purposes of the present invention, “self-foaming” and “foamy” are understood as meaning that the gas bubbles are present in (any) distributed form in one (or more) liquid phase(s), where the preparations do not necessarily have to have the appearance of a foam in macroscopic terms. Self-foaming or foamy cosmetic or dermatological preparations according to the invention may, for example, be macroscopically visibly dispersed systems of gases dispersed in liquids. The foam character can, however, for example, be visible only under a (light) microscope. Moreover, self-foaming or foamy preparations according to the invention are—particularly when the gas bubbles are too small to be recognized under a light microscope—also recognizable from the sharp increase in volume of the system.

The preparations according to the invention are entirely satisfactory preparations in every respect. It was particularly surprising that the foamy preparations according to the invention are extraordinarily stable, even in cases of an unusually high gas volume. Accordingly, they are particularly suitable for use as bases for preparation forms having diverse intended uses. The preparations according to the invention have very good sensory properties, such as, for example, extensibility on the skin or the ability to be absorbed into the skin, and, moreover, are characterized by above-average skincare.

The present invention thus further provides for the use of self-foaming or foamy cosmetic or dermatological preparations which comprise

I. an emulsifier system which consists of

A. at least one emulsifier A chosen from the group of completely neutralized, partially neutralized or unneutralized branched or unbranched, saturated or unsaturated fatty acids with a chain length of from 10 to 40 carbon atoms,

B. at least one emulsifier B chosen from the group of polyethoxylated fatty acid esters with a chain length of from 10 to 40 carbon atoms and with a degree of ethoxylation of from 5 to 100 and

C. at least one coemulsifier C chosen from the group of saturated or unsaturated, branched or unbranched fatty alcohols with a chain length of from 10 to 40 carbon atoms, and

II. up to 30% by weight—based on the total weight of the preparation—of a lipid phase,

IV. 0.01 to 10% by weight of one or more gel formers chosen from the group of inorganic thickeners,

V. one or more substances chosen from the group of organic hydrocolloids,

VI. 0.01 to 10% by weight of one or more particulate hydrophobic or hydrophobicized or oil-absorbing solid-body substances, as cosmetic or dermatological bases for gaseous active ingredients.

The emulsifier(s) A is/are preferably chosen from the group of fatty acids which have been completely or partially neutralized with customary alkalis (such as, for example, sodium hydroxide and/or potassium hydroxide, sodium carbonate and/or potassium carbonate, and mono- and/or triethanolamine). Stearic acid and stearates, isostearic acid and isostearates, palmitic acid and palmitates, and myristic acid and myristates, for example, are particularly advantageous.

The emulsifier(s) B is/are preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Particularly advantageous are, for example, polylethoxylated stearic esters.

The coemulsifier(s) C is/are preferably chosen according to the invention from the following group: butyloctanol, butyldecanol, hexyloctanol, hexyldecanol, octyldodecanol, behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool wax alcohols, which are the unsaponifiable alcohol fraction of wool wax which is obtained following the saponification of wool wax). Particular preference is given to cetyl alcohol and cetylstearyl alcohol.

It is advantageous according to the invention to choose the weight ratios of emulsifier A to emulsifier B to coemulsifier C (A:B:C) as a:b:c, where a, b and c, independently of one another, may be rational numbers from 1 to 5, preferably from 1 to 3. Particular preference is given to a weight ratio of approximately 1:1:1.

It is advantageous for the purposes of the present invention to choose the total amount of emulsifiers A and B and of coemulsifier C from the range from 2 to 20% by weight, advantageously from 5 to 15% by weight, in particular from 8 to 13% by weight, in each case based on the total weight of the formulation.

For the purposes of the present invention, it is particularly preferred if the gas phase of the preparations comprises carbon dioxide or consists entirely of carbon dioxide. It is particularly advantageous if carbon dioxide is a or the active ingredient in the preparations according to the invention.

Compositions according to the invention develop, even during their preparation—for example during stirring or upon homogenization—into fine-bubble foams. According to the invention, fine-bubble, rich foams of excellent cosmetic elegance are obtainable. Furthermore, preparations which are particularly well tolerated by the skin are obtainable according to the invention, where valuable ingredients can be distributed on the skin in a particularly good manner.

It may be advantageous, although it is not necessary, for the formulations according to the present invention to comprise further emulsifiers. Preference is given to using those emulsifiers which are suitable for the preparation of W/O emulsions, it being possible for these to be present either individually or else in any combinations with one another.

The further emulsifier(s) are advantageously chosen from the group which includes the following compounds polyglyceryl-2 dipolyhydroxystearate, PEG-30 dipolyhydroxystearate, cetyldimethicone copolyol, glycol distearate, glycol dilaurate, diethylene glycol dilaurate, sorbitan trioleate, glycol oleate, glyceryl dilaurate, sorbitan tristearate, propylene glycol stearate, propylene glycol laurate, propylene glycol distearate, sucrose distearate, PEG-3 castor oil, pentaerythrityl monostearate, pentaerythrityl sesquioleate, glyceryl oleate, glyceryl stearate, glyceryl diisostearate, pentaerythrityl monooleate, sorbitan sesquioleate, isostearyldiglyceryl succinate, glyceryl caprate, palm glycerides, cholesterol, lanolin, glyceryl oleate (with 40% monoester), polyglyceryl-2 sesquiisostearate, polyglyceryl-2 sesquioleate, PEG-20 sorbitan beeswax, sorbitan oleate, sorbitan isostearate, trioleyl phosphate, glyceryl stearate and ceteareth-20 (Teginacid from Th. Goldschmidt), sorbitan stearate, PEG-7 hydrogenated castor oil, PEG-5 soyasterol, PEG-6 sorbitan beeswax, glyceryl stearate SE, methylglucose sesquistearate, PEG-10 hydrogenated castor oil, sorbitan palmitate, PEG-22/dodecyl glycol copolymer, polyglyceryl-2 PEG-4 stearate, sorbitan laurate, PEG-4 laurate, polysorbate 61, polysorbate 81, polysorbate 65, polysorbate 80, triceteareth-4 phosphate, triceteareth-4 phosphate and sodium C₁₄₋₁₇ alkyl sec sulfonate (Hostacerin CG from Hoechst), glyceryl stearate and PEG-100 stearate (Arlacel 165 from ICI), polysorbate 85, trilaureth-4 phosphate, PEG-35 castor oil, sucrose stearate, trioleth-8 phosphate, C₁₂₋₁₅ pareth-12, PEG-40 hydrogenated castor oil, PEG-16 soya sterol, polysorbate 80, polysorbate 20, polyglyceryl-3 methyl glucose distearate, PEG-40 castor oil, sodium cetearyl sulfate, lecithin, laureth-4 phosphate, propylene glycol stearate SE, PEG-25 hydrogenated castor oil, PEG-54 hydrogenated castor oil, glyceryl stearate SE, PEG-6 caprylic/capric glycerides, glyceryl oleate and propylene glycol, glyceryl lanolate, polysorbate 60, glyceryl myristate, glyceryl isostearate and polyglyceryl-3 oleate, glyceryl laurate, PEG-40 sorbitan peroleate, laureth-4, glycerol monostearate, isostearyl glyceryl ether, cetearyl alcohol and sodium cetearyl sulfate, PEG-22 dodecyl glycol copolymer, polyglyceryl-2 PEG-4 stearate, pentaerythrityl isostearate, polyglyceryl-3 diisostearate, sorbitan oleate and hydrogenated castor oil and Cera alba and stearic acid, sodium dihydroxycetyl phosphate and isopropyl hydroxycetyl ether, methylglucose sesquistearate, methylglucose dioleate, sorbitan oleate and PEG-2 hydrogenated castor oil and ozokerite and hydrogenated castor oil, PEG-2 hydrogenated castor oil, PEG-45/dodecyl glycol copolymer, methoxy PEG-22/dodecyl glycol copolymer, hydrogenated coco glycerides, polyglyceryl-4 isostearate, PEG-40 sorbitan peroleate, PEG-40 sorbitan perisostearate, PEG-8 beeswax, laurylmethicone copolyol, polyglyceryl-2 laurate, stearamidopropyl PG dimonium chloride phosphate, PEG-7 hydrogenated castor oil, triethyl citrate, glyceryl stearate citrate, cetyl phosphate, polyglycerol methylglucose distearate, poloxamer 101, potassium cetyl phosphate, glyceryl isostearate, polyglyceryl-3 diisostearate.

Preferably, for the purposes of the present invention, the further emulsifier(s) is/are chosen from the group of hydrophilic emulsifiers. According to the invention, particular preference is given to mono-, di- and tri-fatty acid esters of sorbitol.

The total amount of further emulsifiers is, according to the invention, advantageously chosen to be less than 5% by weight, based on the total weight of the formulation.

The list of given further emulsifiers which can be used for the purposes of the present invention is not of course intended to be limiting.

Particularly advantageous self-foaming and foamy preparations for the purposes of the present invention are free from mono- or diglyceryl fatty acid esters. Particular preference is given to preparations according to the invention which comprise no glyceryl stearate, glyceryl isostearate, glyceryl diisostearate, glyceryl oleate, glyceryl palmitate, glyceryl myristate, glyceryl lanolate and glyceryl laurate.

The oil phase of the preparations according to the invention is advantageously chosen from the group of nonpolar lipids having a polarity ≧30 mN/m. Particularly advantageous nonpolar lipids for the purposes of the present invention are those listed below.

| | | | Polarity |
|---|---|---|---|
| Manufacturer | Trade name | INCI name | mN/m |
| Total SA | Ecolane 130 | Cycloparaffin | 49.1 |
| Neste PAO N.V. | Nexbase 2006 FG | Polydecene | 46.7 |
| (Supplier Hansen & | | | |
| Rosenthal) | | | |
| Chemische Fabrik | Polysynlane | Hydrogenated | 44.7 |
| Lehrte | | Polyisobutene | |
| Wacker | Wacker Silicone | Polydimethylsiloxane | 46.5 |
| | oil AK 50 | | |
| EC Erdölchemie | Solvent ICH | Isohexadecane | 43.8 |
| (Supplier Bayer AG) | | | |
| DEA Mineral oil (Supplier | Pionier 2076 | Mineral Oil | 43.7 |
| Hansen & Rosenthal) | | | |
| Tudapetrol | | | |
| DEA Mineral oil (Supplier | Pionier 6301 | Mineral Oil | 43.7 |
| Hansen & Rosenthal) | | | |
| Tudapetrol | | | |
| Wacker | Wacker Silicone | Polydimethylsiloxane | 42.4 |
| | oil AK 35 | | |
| EC Erdölchemie GmbH | Isoeicosane | Isoeicosane | 41.9 |
| Wacker | Wacker Silicone | Polydimethylsiloxane | 40.9 |
| | oil AK 20 | | |
| Condea Chemie | Isofol 1212 Carbonate | | 40.3 |
| Gattefossé | Softcutol O | Ethoxydiglycol Oleate | 40.5 |
| Creaderm | Lipodermanol OL | Decyl Olivate | 40.3 |
| Henkel | Cetiol S | Dioctylcyclohexane | 39.0 |
| DEA Mineral oil (Supplier | Pionier 2071 | Mineral Oil | 38.3 |
| Hansen & Rosenthal) | | | |
| Tudapetrol | | | |
| WITCO BV | Hydrobrite 1000 PO | Paraffinum Liquidum | 37.6 |
| Goldschmidt | Tegosoft HP | Isocetyl Palmitate | 36.2 |
| Condea Chemie | Isofol Ester 1693 | | 33.5 |
| Condea Chemie | Isofol Ester 1260 | | 33.0 |
| Dow Corning | Dow Corning Fluid | Cyclopentasiloxane | 32.3 |
| | 245 | | |
| Unichema | Prisorine 2036 | Octyl Isostearate | 31.6 |
| Henkel Cognis | Cetiol CC | Dicaprylyl Carbonate | 31.7 |
| ALZO (ROVI) | Dermol 99 | Trimethylhexyl | 31.1 |
| | | Isononanoate | |
| ALZO (ROVI) | Dermol 89 | 2-Ethylhexyl | 31.0 |
| | | Isononanoate | |
| Unichema | Estol 1540 EHC | Octyl Cocoate | 30.0 |

Of the hydrocarbons, paraffin oil, and further hydrogenated polyolefins, such as hydrogenated polyisobutenes, squalane and squalene, in particular, are to be used advantageously for the purposes of the present invention.

The content of the lipid phase is advantageously chosen to be less than 30% by weight, preferably between 2.5 and 30% by weight, particularly preferably between 5 and 15% by weight, in each case based on the total weight of the preparation. It may also be advantageous, although it is not obligatory, for the lipid phase to comprise up to 40% by weight, based on the total weight of the lipid phase, of polar lipids (having a polarity of ≦20 mN/m) or medium-polarity lipids (having a polarity of from 20 to 30 mN/m).

For the purposes of the present invention, particularly advantageous polar lipids are all native lipids, such as, for example, olive oil, sunflower oil, soybean oil, groundnut oil, rapeseed oil, almond oil, palm oil, coconut oil, castor oil, wheatgerm oil, grapeseed oil, thistle oil, evening primrose oil, macadamia nut oil, corn oil, avocado oil and the like and those listed below.

| | | | Polarity |
|---|---|---|---|
| Manufacturer | Trade name | INCI name | mN/m |
| Condea Chemie | Isofol 14 T | Butyl Decanol (+) Hexyl | 19.8 |
| | | Octanol (+) | |
| | | Hexyl Decanol (+) Butyl | |
| | | Octanol | |
| Lipochemicals | Lipovol MOS-130 | Tridecyl Stearate(+) | 19.4 |
| INC./USA | | Tridecyl | |
| | | Trimellitate(+) | |
| (Induchem) | | Dipentaerythrityl | |
| | | Hexacaprylate/Hexacaprate | |
| | Castor oil | | 19.2 |
| CONDEA | Isofol Ester | | 19.1 |
| Chemie | 0604 | | |
| Huels CONDEA | Miglyol 840 | Propylene Glycol | 18.7 |
| Chemie | | Dicaprylate/Dicaprate | |
| CONDEA Chemie | Isofol 12 | Butyl Octanol | 17.4 |
| Goldschmidt | Tegosoft SH | Stearyl Heptanoate | 17.8 |
| | Avocado oil | | 14.5 |
| Henkel Cognis | Cetiol B | Dibutyl Adipate | 14.3 |
| ALZO (ROVI) | Dermol 488 | PEG 2 Diethylene Hexanoate | 10.1 |
| Condea Augusta | Cosmacol ELI | C₁₂₋₁₃ Alkyl Lactate | 8.8 |
| S.P.A. | | | |
| ALZO (ROVI) | Dermol 489 | Diethylene Glycol | 8.6 |
| | | Dioctanoate(/Diisononanoate | |
| Condea Augusta | Cosmacol ETI | Di-C_{12/13} Alkyl Tartrate | 7.1 |
| S.P.A. | | | |
| Henkel Cognis | Emerest 2384 | Propylene Glycol | 6.2 |
| | | Monoisostearate | |
| Henkel Cognis | Myritol 331 | Cocoglycerides | 5.1 |
| Unichema | Prisorine 2041 | Triisostearin | 2.4 |
| | GTIS | | |

Particularly advantageous medium-polar lipids for the purposes of the present invention are those listed below

| | | | Polarity |
|---|---|---|---|
| | | | (Water) |
| Manufacturer | Trade name | INCI name | mN/m |
| Henkel Cognis | Cetiol OE | Dicaprylyl Ether | 30.9 |
| | Dihexyl carbonate | Dihexyl Carbonate | 30.9 |
| Albemarle S.A. | Silkflo 366 NF | Polydecene | 30.1 |
| Stearinerie | DUB VCI 10 | Isodecyl Neopentanoate | 29.9 |
| Dubois Fils | | | |
| ALZO (ROVI) | Dermol IHD | Isohexyl Decanoate | 29.7 |
| ALZO (ROVI) | Dermol 108 | Isodecyl Octanoate | 29.6 |
| | Dihexyl Ether | Dihexyl Ether | 29.2 |
| ALZO (ROVI) | Dermol 109 | Isodecyl 3,5,5 Trimethyl | 29.1 |
| | | Hexanoate | |
| Henkel Cognis | Cetiol SN | Cetearyl Isononanoate | 28.6 |
| Unichema | Isopropyl | Isopropyl Palmitate | 28.8 |
| | palmitate | | |
| Dow Corning | DC Fluid 345 | Cyclomethicone | 28.5 |
| Dow Corning | Dow Corning | Cyclopolydimethylsiloxane | 28.5 |
| | Fluid 244 | | |
| Nikko Chemicals | Jojoba oil Gold | | 26.2 |
| Superior Jojoba | | | |
| Oil Gold | | | |
| Wacker | Wacker AK 100 | Dimethicone | 26.9 |
| ALZO (ROVI) | Dermol 98 | 2-Ethylhexanoic Acid | 26.2 |
| | | 3,5,5 Trimethyl Ester | |
| Dow Corning | Dow Corning | Open | 25.3 |
| | Fluid 246 | | |
| Henkel Cognis | Eutanol G | Octyldodecanol | 24.8 |
| Condea Chemie | Isofol 16 | Hexyl Decanol | 24.3 |
| ALZO (ROVI) | Dermol 139 | Isotridecyl 3,5,5 | 24.5 |
| | | Trimethylhexanonanoate | |
| Henkel Cognis | Cetiol PGL | Hexyldecanol (+) | 24.3 |
| | | Hexyl Decyl Laurate | |
| | Cegesoft C24 | Octyl Palmitate | 23.1 |
| Gattefossé | M.O.D. | Octyldodeceyl Myristate | 22.1 |
| | Macadamia | | 22.1 |
| | Nut Oil | | |
| Bayer AG, Dow | Silicone oil | Phenyl Trimethicone | 22.7 |
| Corning | VP 1120 | | |
| CONDEA Chemie | Isocarb 12 | Butyl Octanoic Acid | 22.1 |
| Henkel Cognis | Isopropyl | Isopropyl Stearate | 21.9 |
| | stearate | | |
| WITCO, Goldschmidt | Finsolv TN | C12-15 Alkyl Benzoate | 21.8 |
| Dr. Straetmans | Dermofeel BGC | Butylene Glycol | 21.5 |
| | | Caprylate/Caprate | |
| Unichema Huels | Miglyol 812 | Caprylic/Capric | 21.3 |
| | | Triglyceride | |
| Trivent (via | Trivent OCG | Tricaprylin | 20.2 |
| S. Black) | | | |
| ALZO (ROVI) | Dermol 866 | PEG ,, | 20.1 |
| | | Diethylhexanoate/ | |
| | | Diisononanoate/Ethylhexyl | |
| | | Isononanoate | |

The thickener(s) can, for example, advantageously be chosen from the group of modified or unmodified, naturally occurring or synthetic sheet silicates. Although it is entirely favorable to use pure components, the preparations according to the invention may also advantageously comprise mixtures of different modified or unmodified sheet silicates.

Sheet silicates, which are so-called phyllosilicates, are understood for the purposes of this application as meaning silicates and alumosilicates in which the silicate or aluminate units, respectively, are joined together via three Si—O or Al—O bonds and form a waved sheet or layer structure. The fourth Si—O or Al—O valence is saturated by cations. There are relatively weak electrostatic interactions, e.g. hydrogen bridge bonds, between the individual layers. The layer structure is consequently defined largely by strong covalent bonds.

The stoichiometery of the sheet silicates is (Si₂O₃²⁻) for pure silicate structures and (AlₘSi²⁻ₘO₅(^{2+m})⁻) for alumosilicates, wherein m is a number greater than zero and less than 2.

If no pure silicates are present, but alumosilicates, it should be taken into consideration that each Si⁴⁺ group replaced by Al³⁺ requires a further singly charged cation to neutralize the charge.

The charge balance is preferably balanced by H⁺, alkali metal or alkaline earth metal ions. Aluminum as counterion is also known and advantageous. In contrast to the alumosilicates, these compounds are called aluminum silicates. “Aluminum alumosilicates”, in which aluminum is present both in the silicate network, and also as counterion, are also known and in some cases advantageous for the present invention.

Sheet silicates are well documented in the literature, e.g. in the “Lehrbuch der Anorganischen Chemie” [Textbook of inorganic chemistry], A. F. Hollemann, E. Wiberg and N. Wiberg, 91st-100th edition, Walter de Gruyter-Verlag 1985, passim, and also “Lehrbuch der Anorganischen Chemie” [Textbook of inorganic chemistry], H. Remy, 12th edition, Akademische Verlagsgesellschaft, Leipzig 1965, passim. The layer structure of montmorillonite can be found in Römpps Chemie-Lexikon, Franckh'sche Verlagshandlung W. Keller & Co., Stuttgart, 8th edition, 1985, p. 2668 f.

Examples of sheet silicates are:

| | |
|---|---|
| Montmorilonite | Na_{0.33}((Al_{1.67}Mg_{0.33})(OH)₂(Sᵢ₄O₁₀)) |
| often simplified to | Al₂O₃*4SiO₂*H₂O*nH₂O or |
| Kaolinite | Al₂[(OH)₂/Si₄O₁₀] · n H₂O |
| | Al₂(OH)₄(Si₂O₅) |
| Ilite | (K₁H₃O)_{y}(Mg₃(OH)₂(Si_{4-y}Al_{y}O₁₀)) |
| and | (K₁H₃O)_{y}(Al₂(OH)₂(Si_{4-y}Al_{y}O₁₀)) |
| | where y = 0.7-0.9 |
| Beidelite | (Ca,Na)_{0.3}(Al₂(OH)₂(Al_{0.5}Si_{3.5}O₁₀)) |
| Nontronite | Na_{0.33}(Fe₂(OH)₂(Al_{0.33}S_{i3.67}O₁₀)) |
| Saponite | (Ca,Na)_{0.33}(Mg,Fe)₃(OH)₂(Al_{0.33}Si_{3.67}O₁₀)) |
| Hectorite | Na_{0.33}(Mg,Li)₃(OH,F)₂(Si₄O₁₀)) |

Montmorillonite represents the main mineral of the naturally occurring bentonites.

Very advantageous inorganic gel formers for the purposes of the present invention are aluminum silicates, such as the montmorillonites (bentonites, hectorites and derivatives thereof, such as quaternium-18 bentonite, quaternium-18 hectorite, stearalkonium bentonite and stearalkonium hectorite) or however magnesium aluminum silicates (Veegum® grades), and sodium magnesium silicates (Laponite® grades).

Montmorillonites represent clay minerals which belong to the dioctahedral smectites, and are masses which swell in water, but do not become plastic. The layer packets in the three-layer structure of the montmorillonites can swell as the result of reversible incorporation of water (in a 2- to 7-fold amount) and other substances such as, for example, alcohols, glycols, pyridine, α-picoline, ammonium compounds, hydroxy-aluminosilicate ions etc.

The chemical formula given above is only approximate; since montmorillonite has a large capacity for ion exchange, Al can be replaced by Mg, Fe²⁺, Fe³⁺, Zn, Pb, Cr, and also Cu and others. The resulting negative charge of the octahedral layers is balanced by cations, in particular Na⁺ (sodium montmorillonite) and Ca²⁺ (calcium montmorillonite is only swellable to a very small degree) in interlayer positions.

Synthetic magnesium silicates or bentonites advantageous for the purposes of the present invention are sold, for example, by Süd-Chemie under the tradename Optigel®.

An aluminum silicate advantageous for the purposes of the present invention is sold, for example, by R.T. Vanderbilt Comp., Inc., under the trade name Veegum®. The various Veegum® grades, which are all advantageous according to the invention, are characterized by the following compositions

| | (regular | | | | |
|---|---|---|---|---|---|
| | grade) | HV | K | HS | S-728 |
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing loss | 11.1 | 12.6 | 7.6 | 5.5 | 7.5 |

These products swell in water to form viscous gels, which have an alkaline reaction. The organophilization of montmorillonite or bentonites (exchange of the interlayer cations for quaternary alkylammonium ions) produces products (bentones) which are preferably used for dispersion in organic solvents and oils, fats, ointments, paints, coatings and in detergents.

Bentone® is a trade name for various neutral and chemically inert gelling agents which are constructed from long-chain, organic ammonium salts and specific types of montmorillonite. Bentones swell in organic media and cause said media to swell. The gels are stable in dilute acids and alkalis, although they partly lose their gelling properties upon prolonged contact with strong acids and alkalis. Due to their organophilic character, the bentones are only sparingly wettable by water.

The following Bentone® grades are sold, for example, from Kronos Titan: Bentone® 27, an organically modified montmorillonite, Bentone® 34 (dimethyl-dioctylammonium bentonite), which is prepared in accordance with U.S. Pat. No. 2,531,427 and, because of its lipophilic groups, swells more readily in a lipophilic medium than in water, Bentone® 38, an organically modified montmorillonite, a cream-colored to white powder, Bentone® LT, a purified clay mineral, Bentone® Gel MIO, an organically modified montmorillonite which is supplied as a very fine suspension in mineral oil (SUS-71) (10% bentonite, 86.5% mineral oil and 3.3% wetting agent), Bentone® Gel IPM, an organically modified bentonite which is suspended in isopropyl myristate (10% bentonite, 86.7% isopropyl myristate, 3.3% wetting agent), Bentone® Gel CAO, an organically modified montmorillonite which is taken up in castor oil (10% bentonite, 86.7% castor oil and 3.3% wetting agent), Bentone® Gel Lantrol, an organically modified montmorillonite which, in paste form, is intended for the further processing, in particular for the preparation of cosmetic compositions; 10% bentonite, 64.9 Lantrol (wool wax oil), 22.0 isopropyl myristate, 3.0 wefting agent and 0.1 propyl p-hydroxybenzoate, Bentone® Gel Lan I, a 10% strength Bentone® 27 paste in a mixture of wool wax USP and isopropyl palmitate, Bentone® Gel Lan II, a bentonite paste in pure, liquid wool wax, Bentone® Gel NV, a 15% strength Bentone® 27 paste in dibutyl phthalate, Bentone® Gel OMS, a bentonite paste in Shellsol T., Bentone® Gel OMS 25, a bentonite paste in isoparaffinic hydrocarbons (Idopar® H), Bentone® Gel IPP, a bentonite paste in isopropyl palmitate.

All Bentone grades are to be used advantageously for the purposes of the present invention.

“Hydrocolloid” is the technological abbreviation for the more correct name “hydrophilic colloid”. Hydrocolloids are macromolecules which have a largely linear structure and have intermolecular forces of interaction which permit secondary and primary valence bonds between the individual molecules and thus the formation of a reticular structure. Such water-soluble polymers represent a large group of chemically very different natural and synthetic polymers whose common feature is their solubility in water or aqueous media. A prerequisite for this is that these polymers have a large number of hydrophilic groups sufficient for solubility in water and are not too greatly crosslinked. The hydrophilic groups may be nonionic, anionic or cationic in nature, for example as follows:

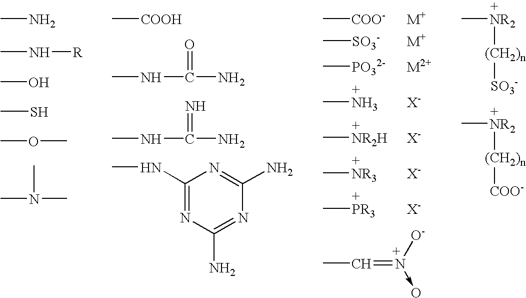

The group of the cosmetically and dermatologically relevant hydrocolloids can be divided as follows into:

organic, natural compounds, such as, for example, agar agar, carrageen, tragacanth, gum arabic, alginates, pectins, polyoses, guar flour, carob bean flour, starch, dextrins, gelatin, casein,

organic, modified natural substances, such as, for example, carboxymethylcellulose and other cellulose ethers, hydroxyethyl cellulose and hydroxypropyl cellulose and microcrystalline celluose the like,

organic, completely synthetic compounds, such as, for example, polyacrylic and polymethacrylic compounds, vinyl polymers, polycarboxylic acids, polyethers, polyimines, polyamides, polylurethanes.

Microcrystalline cellulose is an advantageous hydrocolloid for the purposes of the present invention. It is obtainable, for example, from the “FMC Corporation Food and Pharmaceutical Products” under the trade name Avicel®. A particularly advantageous product for the purposes of the present invnetion is the Avicel® grade RC-591, which is modified microcrystalline cellulose which is composed of 89% of microcrystalline cellulose and 11% of sodium carboxymethylcellulose. Further commercial products of this class of raw material are Avicel® RC/CL, Avicel® CE-15, Avicel® 500.

Further hydrocolloids which are advantageous according to the invention are, for example, methylcelluloses, which is the term used for the methyl ethers of cellulose. They are characterized by the following structural formula

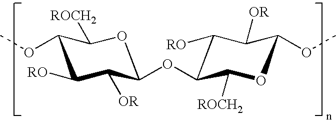

in which R may be a hydrogen or a methyl group.

Particularly advantageous for the purposes of the present invention are the cellulose mixed ethers, which are generally likewise referred to as methylcelluloses, which contain, in addition to a predominating content of methyl groups, additionally 2-hydroxyethyl groups, 2-hydroxypropyl groups or 2-hydroxybutyl groups. Particular preference is given to (hydroxypropyl)methylcelluloses, for example those available under the trade name Methocel® E4M from Dow Chemical Comp.

Also advantageous according to the invention is sodium carboxymethylcellulose, the sodium salt of the glycolic ether of cellulose, for which R in structural formula I may be a hydrogen or CH₂—COONa. Particular preference is given to the sodium carboxymethylcellulose available under the trade name Natrosol Plus 330 CS from Aqualon and also referred to as cellulose gum.

A preferred hydrocolloid for the purposes of the present invention is also xanthan (CAS No. 11138-66-2), also called xanthan gum, which is an anionic heteropolysaccharide which is usually formed by fermentation from corn sugar and is isolated as the potassium salt. It is produced by Xanthomonas campestris and some other species under aerobic conditions and has a molecular weight of from 2×10⁶ to 24×10⁶. Xanthan is formed from a chain having β-1,4-bonded glucose (cellulose) with side chains. The structure of the subgroups consists of glucose, mannose, glucuronic acid, acetate and pyruvate. Xanthan is the name given to the first microbial anionic heteropolysaccharide. It is produced by Xanthomonas campestris and some other species under aerobic conditions and has a molecular weight of from 2-15 10⁶. Xanthan is formed from a chain having β-1,4-bonded glucose (cellulose) with side chains. The structure of the subgroups consists of glucose, mannose, glucuronic acid, acetate and pyruvate. The number of pyruvate units determines the viscosity of the xanthan.

An advantageous hydrocolloid for the purposes of the present invention is also carrageen, a gel-forming extract with a similar structure to agar, from North Atlantic red algae, which belong to the Florideae (Chondrus crispus and Gigartina stellata).

The term carrageen is frequently used for the dried algae product and carrageenan for the extract thereof. The carrageen precipitated from the hot-water extract of the algae is a colorless to sand-colored powder with a molecular weight range from 100 000-800 000 and a sulfate content of about 25%. Carrageen, which is very readily soluble in warm water, forms a thixotropic gel upon cooling, even if the water content is 95-98%. The rigidity of the gel is effected by the double helix structure of carrageen. In the case of carrageenan, three main constituents are differentiated: the gel-forming K fraction consists of D-galactose 4-sulfate and 3,6-anhydro-α-D-galactose, which has alternate glycoside bonds in the 1,3- and 1,4-position (by contrast, agar contains 3,6-anhydro-α-L-galactose). The nongelling λ fraction is composed of 1,3-glycosidically linked D-galactose 2-sulfate and 1,4-bonded D-galactose-2,6-disulfate radicals, and is readily soluble in cold water. i-Carrageenan, composed of D-galactose 4-sulfate in 1,3 bond and 3,6-anhydro-α-D-galactose 2-sulfate in 1,4 bond, is both water-soluble and also gel-forming. Further carrageen grades are likewise referred to using Greek letters: α, β, γ, μ, ν, ζ, π, ω, χ. The type of cations present (K⁺, NH₄⁺, Na⁺, Mg²⁺, Ca²⁺) also influences the solubility of the carrageens.

Chitosan is also an advantageous hydrocolloid for the purposes of the present invention. The use of chitosan in cosmetic preparations is known per se. Chitosan represents a partially deacylated chitin. This biopolymer has, inter alia, film-forming properties and is characterized by a silky feel on the skin. As is known, chitosan is used, for example, in hair care. It is suitable, to a better degree than the chitin on which it is based, as a thickener or stabilizer and improves the adhesion and water resistance of polymeric films. A representative of a large number of literature references for the prior art is: H. P. Fiedler, “Lexikon der Hilfsstoffe für Pharmazie, Kosmetik und angren-zende Gebiete” [Lexikon of Auxiliaries for Pharmacy, Cosmetics and Related Fields], third edition 1989, Editio Cantor, Aulendorf, p. 293, key word “chitosan”.

Chitosan is characterized by the following structural formula:

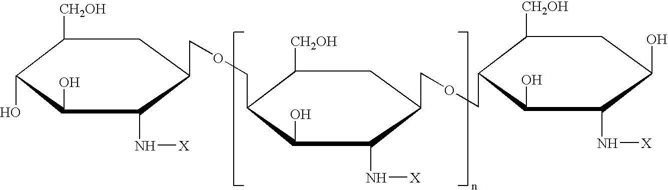

where n assumes values of up to 10 000, and X is either the acetyl radical or hydrogen. Chitosan forms by deacetylation and partial depolymerization (hydrolysis) of chitin, which is characterized by the structural formula

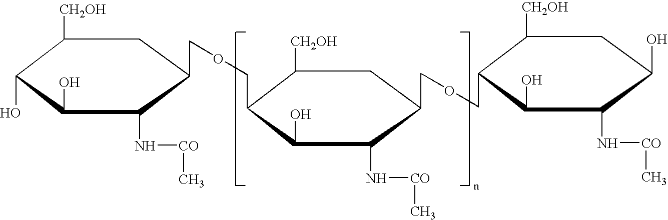

Chitin is an essential constituent of the ectoskeleton of arthropods (e.g. insects, crabs, spiders) and is also found in supporting tissues of other organisms (e.g. molluscs, algae, fungi).

In the region of about pH <6, chitosan is positively charged and in that range is also soluble in aqueous systems. It is incompatible with anionic raw materials. For this reason, to prepare chitosan-containing oil-in-water emulsions, the use of nonionic emulsifiers is appropriate. These are known per se, for example from EP-A 776 657.

Preference is given according to the invention to chitosans with a degree of deacetylation of >25%, in particular >55 to 99% [determined by means of ¹H-NMR]).

It is advantageous to choose chitosans with molecular weights between 10 000 and 1 000 000, in particular those with molecular weights between 100 000 and 1 000 000 [determined by means of gel permeation chromatography].

Polyacrylates are hydrocolloids likewise to be used advantageously for the purposes of the present invention. Polyacrylates advantageous according to the invention are acrylate-alkyl acrylate copolymers, in particular those chosen from the group of so-called carbomers or carbopols (Carbopol® is actually a registered trademark of B. F. Goodrich Company). In particular, the acrylate-alkyl acrylate copolymers advantageous according to the invention are characterized by the following structure:

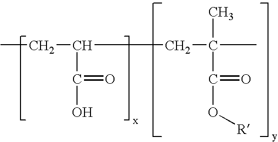

where R′ is a long-chain alkyl radical, and x and y represent numbers which symbolize the respective stoichiometric proportion of each of the comonomers.

According to the invention, particular preference is given to acrylate copolymers or acrylate-alkyl acrylate copolymers which are available under the trade names Carbopol® 1382, Carbopol® 981 and Carbopol® 5984 from B. F. Goodrich Company, preferably polyacrylates from the group of Carbopol grades 980, 981, 1382, 2984, 5984 and particularly preferably Carbomer 2001

Also advantageous for the purposes of the present invention are the copolymers, comparable with the acrylate-alkyl acrylate copolymers, of C₁₀₋₃₀-alkyl acrylates and one or more monomers of acrylic acid, of methacrylic acid or esters thereof. The INCI name for such compounds is “Acrylates/C 10-30 Alkyl Acrylate Crosspolymer”. Those available under the trade names Pemulen TR1 and Pemulen TR2 from B.F. Goodrich Company are particularly advantageous.

Also advantageous for the purposes of the present invention are compounds which have the INCI name Ammonium acryloyidimethyltaurate/Vinylpyrrolidone copolymers.

Advantageously according to the invention, the ammonium acryloyidimethyltaurates/vinylpyrrolidone copolymers have the empirical formula [C₇H₁₆N₂SO₄]ₙ[C₆H₉NO]ₘ, which corresponds to the following statistical structure

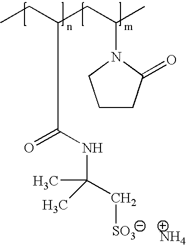

Preferred species for the purposes of the present invention are listed in the Chemical Abstracts under the registry numbers 58374-69-9, 13162-05-5 and 88-12-0 and are available under the trade name Aristoflex® AVC from Clariant GmbH.

Also advantageous are copolymers/crosspolymers comprising acryloyl dimethyl taurate, such as, for example, Simugel® EG or Simugel® EG from Seppic S.A.

Further hydrocolloids which are to be used advantageously according to the invention are also

1. anionic polyurethanes which are soluble or dispersible in water and which are advantageously obtainable from

(i) at least one compound which contains two or more active hydrogen atoms per molecule,

(ii) at least one diol containing acid or salt groups and

(iii) at least one diisocyanate.

Component (i) is, in particular, a diol, aminoalcohol, diamine, polyesterol, polyetherol with a number-average molecular weight of in each case up to 3000, or mixtures thereof, where up to 3 mol% of said compounds may be replaced by triols or triamines. Preference is given to diols and polyesterdiols. In particular, component (i) comprises at least 50% by weight, based on the total weight of component (i), of a polyesterdiol. Suitable polyesterdiols are all those which are customarily used for the preparation of polyurethanes, in particular reaction products of phthalic acid and diethylene glycol, isophthalic acid and 1,4-butanediol, isophthalic acid/adipic acid and 1,6-hexanediol, and adipic acid and ethylene glycol or 5-NaSO₃-isophthalic acid, phthalic acid, adipic acid and 1,6-hexanediol.

Examples of diols which can be used are ethylene glycol, propylene glycol, butylene glycol, neopentyl glycol, polyetherols, such as polyethylene glycols with molecular weights up to 3000, block copolymers of ethylene oxide and propylene oxide with number-average molecular weights of up to 3000 or block copolymers of ethylene oxide, propylene oxide and butylene oxide which contain the copolymerized alkylene oxide units in random distribution or in the form of blocks. Preference is given to ethylene glycol, neopentyl glycol, di-, tri-, tetra-, penta- or hexaethylene glycol. Other diols which can be used are poly(α-hydroxycarboxylic acid)diols.

Suitable amino alcohols are, for example, 2-aminoethanol, 2-(N-methylamino)ethanol, 3-aminopropanol or 4-aminobutanol.

Examples of suitable diamines are ethylenediamine, propylenediamine, 1,4-diaminobutane and 1,6-diaminohexane, and α,ω-diamines which can be prepared by amination of polyalkylene oxides with ammonia.

Component (ii) is, in particular, dimethylolpropanoic acid or compounds of the formulae

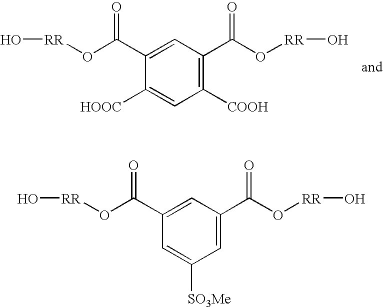

where RR is in each case a C₂-C₁₈-alkylene group and Me is Na or K.

Component (iii) is, in particular, hexamethylene diisocyanate, isophorone diisocyanate, methyidiphenyl isocyanate (MDI) or tolylene diisocyanate.

The polyurethanes are obtainable by reacting the compounds of groups (i) and (ii) under an inert-gas atmosphere in an inert solvent at temperatures of from 70 to 130° C. with the compounds of group (iii). This reaction can optionally be carried out in the presence of chain extenders in order to prepare polyurethanes with relatively high molecular weights. As is customary in the preparation of polyurethanes, the components [(i)+(ii)]:(iii) are advantageously used in the molar ratio from 0.8 to 1.1:1. The acid number of the polyurethanes is determined by the composition and the concentration of the compounds of component (ii) in the mixture of components (i)+(ii).

The polyurethanes have K values according to H. Fikentscher (determined in 0.1% strength by weight solutions in N-methylpyrrolidone at 25° C. and pH 7) of from 15 to 100, preferably 25 to 50.

The K value, also referred to as the intrinsic viscosity, is a parameter which is easy to determine by means of viscosity measurements of polymer solutions and is therefore frequently used in the industrial sector for characterizing polymers. For a certain sort of polymer, it is accepted under standardized measurement conditions on its own, depending on the average molar mass of the investigated sample and is calculated by means of the equation K value=1000 k according to the Fikentscher equation

$$k = \frac{1.5 lg {η}_{r} - 1 \pm \sqrt{1 + ( \frac{2}{c} + 2 + 1.5 lg {η}_{r} ) \cdot 1.5 lg {η}_{r}} }{150 + 300 c}$$

in which: ηᵣ=relative viscosity (dynamic viscosity of the solution/dynamic viscosity of the solvent) and c=mass concentration of polymer in the solution (in g/cm³).

The polyurethanes containing acid groups are, after neutralization (partial or complete), water-soluble or dispersible without the aid of emulsifiers. The salts of the polyurethanes generally have better solubility or dispersibility in water than the unneutralized polyurethanes. Bases which can be used for the neutralization of the polyurethanes are alkali metal bases, such as sodium hydroxide solution, potassium hydroxide solution, soda, sodium hydrogencarbonate, potassium carbonate or potassium hydrogencarbonate, and alkaline earth metal bases, such as calcium hydroxide, calcium oxide, magnesium hydroxide or magnesium carbonate, and ammonia and amines. 2-Amino-2-methylpropanol, diethylaminopropylamine and triisopropanolamine have proven particularly useful for the neutralization of the polyurethanes containing acid groups. The neutralization of the polyurethanes containing acid groups can also be carried out using mixtures of two or more bases, e.g. mixtures of sodium hydroxide solution and triisopropanolamine. Depending on the intended use, neutralization may be partial, e.g. up to 20 to 40%, or complete, i.e. 100%.

These polymers and their preparation are described in more detail in DE-A-42 25 045, to the entire scope of which reference is hereby made.

2. Water-soluble or -dispersible cationic polyurethanes and polyureas comprising

(a) at least one diisocyanate, which may have already been reacted beforehand with one or more compounds which contain two or more active hydrogen atoms per molecule, and

(b) at least one diol, primary or secondary aminoalcohol, primary or secondary diamine or primary or secondary triamine with one or more tertiary, quaternary or protonated tertiary amino nitrogen atoms.

Preferred diisocyanates are as given above under 1. Compounds with two or more active hydrogen atoms are diols, aminoalcohols, diamines, polyesterols, polyamidediamines and polyetherols. Suitable compounds of this type are as given above under 1.

The polyurethanes are prepared as described above under 1. Charged cationic groups can be produced in the polyureas from the tertiary amino nitrogen atoms present either by protonation, e.g. with carboxylic acids, such as lactic acid, or by quaternization, e.g. with alkylating agents, such as C₁-C₄-alkyl halides, or sulfates. Examples of such alkylating agents are ethyl chloride, ethyl bromide, methyl chloride, methyl bromide, dimethyl sulfate and diethyl sulfate.

These polymers and their preparation are described in more detail in DE-A-42 41 118, to the entire scope of which reference is hereby made.

3. Linear polyurethanes with carboxylate groups comprising

(i) a 2,2-hydroxymethyl-substituted carboxylic acid of the formula

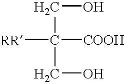

in which RR′ is a hydrogen atom or a C₁-C₂₀-alkyl group, which is used in an amount which suffices for 0.35 to 2.25 milliequivalents of carboxyl groups to be present in the polyurethane per g of polyurethane,

(ii) 10 to 90% by weight, based on the weight of the polyurethane, of one or more organic compounds with not more than two active hydrogen atoms and

(iii) one or more organic diisocyanates.

The carboxyl groups present in the polyurethane are, finally, at least partially neutralized with a suitable base. These polymers and their preparation are described in EP-A-619 111, to the entire scope of which reference is hereby made.

4. Carboxyl-containing polycondensation products of anhydrides of tri- or tetracarboxylic acids and diols, diamines or amino alcohols (polyesters, polyamides or polyesteramides). These polymers and their preparation are described in more detail in DE-A-42 24 761, to the entire scope of which reference is hereby made.

5. Polyacrylates and polymethacrylates, as are described in more detail in DE-A-43 14 305, 36 27 970 and 29 17 504. Reference is hereby made to these publications in their entirety.

The polymers used according to the invention preferably have a K value of from 25 to 100, preferably 25 to 50. The polymers are generally present in the composition according to the invention in an amount in the range from 0.2 to 20% by weight, based on the total weight of the composition. The salt is used in an amount effective for improving the exchangeability of the polymers. In general, the salt is used in an amount of from 0.02 to 10% by weight, preferably 0.05 to 5% by weight and in particular 0.1 to 3% by weight, based on the total weight of the composition.

For the purposes of the present invention, the hydrocolloids can also, for example, be advantageously chosen from the group of modified carbohydrate derivatives, starch and starch derivatives (distarch phosphate, sodium and aluminum starch octenylsuccinate, wheat starch, corn starch (Amidon De Mais MST (Wackherr), Argo brand corn starch (Corn Products), Pure-Dent (Grain Processing), Purity 21C (National Starch), rice starch (D.S.A. 7 (Agrana Starch), Oryzapearl (Ichimaru Pharcos), hydoxypropyl starch phosphate distarch phosphate (Corn PO4 (Agrana Starch) Corn PO4 (Tri-K)) sodium corn starch octenylsuccinate (C* EmCap—Instant 12639 (Cerestar USA)) aluminum starch octenylsuccinate (Covafluid AMD (Wackherr) Dry Flo-PC (National Starch) Dry Flo Pure (National Starch) Fluidamide DF 12 (Roquette)).

The total amount of one or more hydrocolloids in the finished cosmetic or dermatological preparations is advantageously chosen to be less than 5% by weight, preferably between 0.1 and 1.0% by weight, based on the total weight of the preparations.

The inorganic particulate hydrophobic or hydrophobicized or oil-absorbing solid-body substances may, for example, advantageously be chosen from the group

of inorganic fillers (such as talc, kaolin, zeolites, boron nitride)

of inorganic pigments based on metal oxides or other metal compounds which are sparingly soluble or insoluble in water (in particular oxides of titanium, zinc, iron, manganese, aluminum, cerium)

of inorganic pigments based on silicon oxides (such as, in particular, the grades Aerosil-200, Aerosil 200 V)

of silicate derivatives (such as sodium silicoaluminates or fluoro magnesium silicates (submica grades), calcium aluminum borosilicates). Preference is given here in particular to silica dimethyl silylate (Aerosil® R972).

Further oil-absorbing solid-body substances advantageous according to the invention are microspherical particles which are based on crosslinked polymethyl methacrylates (INCI: Crosslinked Methyl Methacrylates). These are sold by SEPPIC under the trade names Micropearl® M305, Micropearl® 201, Micropearl® M 310 and Micropearl® MHB and are characterized by an oil-absorption capacity of 40-100 g/100 g.

Aerosils (fumed silica)=silicon dioxide obtained by thermal decomposition of ethyl silicate) are highly disperse silicas with an often irregular shape, whose specific surface area is usually very large (200-400 m²/g) and can be controlled depending on the preparation process.

Aerosils to be used advantageously according to the invention are obtainable, for example, under the trade names: Aerosil® 130 (Degussa Hüls) Aerosil® 200 (Degussa Hüls) Aerosil® 225 (Degussa Hüls) Aerosil® 300 (Degussa Hüls) Aerosil® 380 (Degussa Hüls) B-6C (Suzuki Yushi) CAB-O-SIL Fumed Silica (Cabot) CAB-O-SIL EH-5 (Cabot) CAB-O-SIL HS-5 (Cabot) CAB-O-SIL LM-130 (Cabot) CAB-O-SIL MS-55 (Cabot) CAB-O-SIL M-5 (Cabot) E-6C (Suzuki Yushi) Fossil Flour MBK (MBK) MSS-500 (Kobo) Neosil CT 11 (Crosfield Co.) Ronasphere (Rona/EM Industries) Silica, Anhydrous 31 (Whittaker, Clark & Daniels) Silica, Crystalline 216 (Whittaker, Clark & Daniels) Silotrat-1 (Vevy) Sorbosil AC33 (Crosfield Co.) Sorbosil AC 35 (Crosfield Co.) Sorbosil AC 37 (Crosfield Co.) Sorbosil AC 39 (Crosfield Co.) Sorbosil AC77 (Crosfield Co.) Sorbosil TC 15 (Crosfield Co.) Spherica (Ikeda) Spheriglass (Potters-Ballotini) Spheron L-1500 (Presperse) Spheron N-2000 (Presperse) Spheron P-1500 (Presperse) Wacker HDK H 30 (Wacker-Chemie) Wacker HDK N 20 (Wacker-Chemie) Wacker HDK P 100 H (Wacker Silicones) Wacker HDK N 20P (Wacker-Chemie) Wacker HDK N 25P (Wacker-Chemie) Wacker HDK S 13 (Wacker-Chemie) Wacker HDK T 30 (Wacker-Chemie) Wacker HDK V 15 (Wacker-Chemie) Wacker HDK V 15 P (Wacker Chemie) Zelec Sil (DuPont).

It is also advantageous to use those SiO₂ pigments in which the free OH groups on the surface of the particles have been (completely or partially) organically modified. This gives, for example as a result of the addition of dimethylsilyl groups, silica dimethyl silylate (e.g. Aerosil® R972 (Degussa Hüls) Aerosil® R974 (Degussa Hüls) CAB-O-SIL TS-610 (Cabot) CAB-O-SIL TS-720 (Cabot) Wacker HDK H15 (Wacker-Chemie) Wacker HDK H18 (Wacker-Chemie) Wacker HDK H20 (Wacker-Chemie)). The addition of trimethylsilyl groups gives silica silylate (e.g. Aerosil R 812 (Degussa Hüls) CAB-O-SIL TS-530 (Cabot) Sipernat D 17 (Degussa Hüls) Wacker HDK H2000 (Wacker-Chemie)).

Polymethylsilsesquioxanes are supplied, for example, under the trade names Tospearl® 2000 B from GE Bayer Silikones, Tospearl 145A from Toshiba, AEC Silicone Resin Spheres from A & E Connock or Wacker—Belsil PMS MK from Wacker-Chemie.

The cosmetic and dermatological preparations according to the invention can have the customary composition. Particularly advantageous for the purposes of the present invention are skincare preparations: they can be used for cosmetic or dermatological light protection, and also for the treatment of the skin or of the hair and as a make-up product in decorative cosmetics. A further advantageous embodiment of the present invention consists in after-sun products.

Depending on their formulation, cosmetic or topical dermatological compositions for the purposes of the present invention can, for example, be used as skin protection cream, day or night cream etc. It is optionally possible and advantageous to use the compositions according to the invention as bases for pharmaceutical formulations.

Just as emulsions of liquid and solid consistency are used as cosmetic cleansing lotions or cleansing creams, the preparations according to the invention can also represent “cleansing foams”, which can be used, for example, for the removal of make-up or as a mild washing foam—optionally also for blemished skin. Such cleansing foams can also advantageously be applied as so-called “rinse off” preparations, which are rinsed off from the skin following application.

The cosmetic and dermatological preparations according to the invention can also advantageously be in the form of a foam for the care of hair or the scalp, in particular a foam for arranging the hair, a foam which is used while blow-drying the hair, a styling and treatment foam.

For use, the cosmetic and dermatological preparations according to the invention are applied to the skin or the hair in an adequate amount in the manner customary for cosmetics.

The cosmetic and dermatological preparations according to the invention can comprise cosmetic auxiliaries, as are customarily used in such preparations, e.g. preservatives, preservative assistants, bactericides, perfumes, dyes, pigments which have a coloring effect, moisturizing or humectant substances, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin), iodopropyl butylcarbamates (e.g. those available under the trade names Koncyl-L, Koncyl-s and Konkaben LMB from Lonza), parabens, phenoxyethanol, ethanol, benzoic acid and the like. According to the invention, the preservative system usually also advantageously comprises preservative assistants, such as, for example, octoxyglycerol, glycine soya etc.

Particularly advantageous preparations are also obtained if antioxidants are used as additives or active ingredients. According to the invention, the preparations advantageously comprise one or more antioxidants. Favorable, but nevertheless optional antioxidants which may be used are all antioxidants customary or suitable for cosmetic and dermatological applications.

For the purposes of the present invention, water-soluble antioxidants, such as, for example, vitamins, e.g. ascorbic acid and derivatives thereof, can be used particularly advantageously.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E or derivatives thereof are the antioxidant(s), it is advantageous to choose their respective concentrations from the range from 0.001 to 10% by weight, based on the total weight of the formulation.

If vitamin A or vitamin A derivatives, or carotenes or derivatives thereof are the antioxidant(s), it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients or derivatives thereof, such as, for example, alpha-lipoic acid, phytoene, D-biotin, coenzyme Q10, alpha-glycosylrutin, carnitine, carnosine, natural or synthetic isoflavonoids, creatine, taurine or β-alanine.

Formulations according to the invention which comprise, for example, known antiwrinkle active ingredients, such as flavone glycosides (in particular α-glycosylrutin), coenzyme Q10, vitamin E or derivatives and the like are particularly advantageously suitable for the prophylaxis and treatment of cosmetic or dermatological changes in the skin, as arise, for example, during skin aging (such as, for example, dryness, roughness and the formation of dryness wrinkles, irritation, reduced refatting (e.g. after washing), visible vascular dilations (telangiectases, couperosis), sagging and formation of lines and wrinkles, local hyperpigmentation, hypopigmentation and abnormal pigmentation (e.g. age spots), increased susceptibility to mechanical stress (e.g. cracking) and the like. They are also advantageously suitable against the appearance of dry or rough skin.

Surprisingly, selected formulations according to the invention can also have an antiwrinkle effect or considerably increase the effect of known antiwrinkle active ingredients. Accordingly, formulations for the purposes of the present invention are particularly advantageously suitable for the prophylaxis and treatment of cosmetic or dermatological skin changes, as arise, for example, during skin aging. They are also advantageously suitable for combating the appearance of dry or rough skin.

In one particular embodiment, the present invention thus relates to products for the care of skin aged in a natural manner, and for the treatment of the secondary damage of photoaging, in particular the phenomena listed above.

The water phase of the preparations according to the invention can advantageously comprise customary auxiliaries, such as, for example, alcohols, in particular those of low carbon number, preferably ethanol or isopropanol, diols or polyols of low carbon number, and ethers thereof, preferably polyethylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and moisturizers.

Moisturizers is the term used to describe substances or mixtures of substances which, following application or distribution on the surface of the skin, confer on cosmetic or dermatological preparations the property of reducing the moisture loss by the horny layer (also called transepidermal water loss (TEWL) or have a beneficial effect on the hydration of the horny layer.

Advantageous moisturizers for the purposes of the present invention are, for example, glycerol, lactic acid, pyrrolidone carboxylic acid and urea. In addition, it is particularly advantageous to use polymeric moisturizers from the group of water-soluble or water-swellable or water-gelable polysaccharides. Particularly advantageous are, for example, hyaluronic acid, chitosan or a fucose-rich polysaccharide which is listed in the Chemical Abstracts under the registry number 178463-23-5 and is available, for example, under the name Fucogel® 1000 from SOLABIA S.A.

The cosmetic and dermatological preparations according to the invention can comprise dyes or color pigments, particularly when they are in the form of decorative cosmetics. The dyes and color pigments can be chosen from the corresponding positive list of the Cosmetics Directive or the EC list of cosmetic colorants. In most cases they are identical to the dyes approved for foodstuffs. Advantageous color pigments are, for example, titanium dioxide, mica, iron oxides (e.g. Fe₂O₃, Fe₃O₄, FeO(OH)) and tin oxide. Advantageous dyes are, for example, carmine, Prussian blue, chromium oxide green, ultramarine blue and manganese violet. It is particularly advantageous to choose the dyes or color pigments from the Rowe Colour Index, 3rd edition, Society of Dyers and Colourists, Bradford, England, 1971.

If the formulations according to the invention are in the form of products which are used on the face, it is favorable to choose one or more substances from the following group: 2,4-dihdyroxyazobenzene, 1-(2′-chloro-4′-nitro-1′-phenylazo)-2-hydroxynaphthalene, Ceres red, 2-(sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salts, aluminum salt of 2,4,5,7-tetraiodofluorescein, aluminum salt of quinophthalonedisulfonic acid, aluminum salt of indigodisulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochineal.

Also advantageous for the purposes of the present invention are formulations with a content of pearlescent pigments. Preference is given in particular to the types of pearlescent pigments listed below:

1. Natural pearlescent pigments, such as, for example

“pearl essence” (guanine/hypoxanthin mixed crystals from fish scales) and

“mother of pearl” (ground mussel shells)

2. Monocrystalline pearlescent pigments, such as, for example, bismuth oxychloride (BiOCl)

3. Layer-substrate pigments: e.g. mica/metal oxide

Bases for pearlescent pigments are, for example, pulverulent pigments or castor oil dispersions of bismuth oxychloride or titanium dioxide, and bismuth oxychloride or titanium dioxide on mica. The luster pigment listed under CIN 77163, for example, is particularly advantageous.

Also advantageous are, for example, the following types of pearlescent pigment based on mica/metal oxide:

| | Coating/layer | |
|---|---|---|
| Group | thickness | Color |
| Silver-white | TiO₂: 40-60 nm | silver |
| pearlescent | | |
| pigments | | |
| Interference | TiO₂: 60-80 nm | yellow |
| pigments | | |
| | TiO₂: 80-100 nm | red |
| | TiO₂: 100-140 nm | blue |
| | TiO₂: 120-160 nm | green |
| Color luster | Fe₂O₃ | bronze |
| pigments | Fe₂O₃ | copper |
| | Fe₂O₃ | red |
| | Fe₂O₃ | red-violet |
| | Fe₂O₃ | red-green |
| | Fe₂O₃ | black |
| Combination | TiO₂/Fe₂O₃ | gold shades |
| pigments | TiO₂/Cr₂O₃ | green |
| | TiO₂/Prussian blue | deep blue |
| | TiO₂/carmine | red |

Particular preference is given, for example, to the pearlescent pigments obtainable from Merck under the trade names Timiron, Colorona and Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are marketed by Merck and are particularly suitable for the optical reduction of fine lines are advantageous.

It may, moreover, be advantageous to dispense completely with a substrate such as mica. Particular preference is given to iron pearlescent pigments prepared without the use of mica. Such pigments are obtainable, for example, under the trade name Sicopearl Copper 1000 from BASF.

In addition, also particularly advantageous are effect pigments which are obtainable under the trade name Metasomes Standard/Glitter in various colors (yellow, red, green, blue) from Flora Tech. The glitter particles here are present in mixtures with various auxiliaries and dyes (such as, for example, the dyes with the Colour Index (CI) numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from, for example, 0.1% by weight to 30% by weight, preferably from 0.5 to 15% by weight, in particular from 1.0 to 10% by weight, in each case based on the total weight of the preparations.

For the purposes of the present invention, it is also advantageous to provide cosmetic and dermatological preparations whose main purpose is not protection against sunlight, but which nevertheless have a content of UV protectants. Thus, for example, UV-A or UV-B filter substances are usually incorporated into day creams or make-up products. UV protectants, like antioxidants, and, if desired, preservatives, also constitute effective protection of the preparations themselves against spoilage. Also favorable are cosmetic and dermatological preparations in the form of a sunscreen.

Accordingly, for the purposes of the present invention, besides comprising one or more UV filter substances according to the invention, the preparations additionally comprise at least one further UV-A or UV-B filter substance. The formulations may, although not necessarily, optionally also comprise one or more organic or inorganic pigments as UV filter substances which may be present in the water or oil phase.

The preparations according to the invention can, in addition, also advantageously be in the form of so-called oil-free cosmetic or dermatological emulsions which comprise a water phase and at least one UV filter substance which is liquid at room temperature or one or more silicone derivatives as a further phase. Oil-free formulations for the purposes of the present invention can advantageously also comprise further lipophilic components—such as, for example, lipophilic active ingredients.

Particularly advantageous UV filter substances which are liquid at room temperature for the purposes of the present invention are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Octyl Salicylate) and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate).

Preferred inorganic pigments are metal oxides or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and also the sulfate of barium (BaSO₄).

The pigments can, for the purposes of the present invention, also advantageously be used in the form of commercially available oily or aqueous predispersions. Dispersion auxiliaries or solubilization promoters may advantageously be added to these predispersions.

According to the invention, the pigments may advantageously by surface-treated (“coated”), the intention being to form or retain, for example, an amphiphilic or hydrophobic character. This surface treatment can consist in providing the pigments with a thin hydrophilic or hydrophobic inorganic or organic layer by processes known per se. The various surface coatings may also comprise water for the purposes of the present invention.

Inorganic surface coatings for the purposes of the present invention may consist of aluminum oxide (Al₂O₃), aluminum hydroxyide Al(OH)₃, and aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings can arise on their own or in combination or in combination with organic coating materials.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination or in combination with inorganic coating materials.

Zinc oxide particles suitable according to the invention and predispersions of zinc oxide particles are obtainable under the following trade names from the companies listed:

| Trade name | Coating | Manufacturer |
|---|---|---|
| Z - Cote HP1 | 2% dimethicone | BASF |
| Z - Cote | / | BASF |
| ZnO NDM | 5% dimethicone | H&R |

Suitable titanium dioxide particles and predispersions of titanium dioxide particles are obtainable under the following trade names from the companies listed:

| Trade name | Coating | Manufacturer |
|---|---|---|
| MT-100TV | aluminum hydroxide/stearic | Tayca Corporation |
| | acid | |
| MT-100Z | aluminum hydroxide/stearic | Tayca Corporation |
| | acid | |
| Eusolex T-2000 | alumina/simethicone | Merck KgaA |
| Titanium dioxide | octyltrimethylsilane | Degussa |
| T805 (Uvinul TiO₂) | | |

An advantageous organic pigment for the purposes of the present invention is 2,2′-methylenebis(6-(2H-benzotriazol-2-yl)-4-(1,1,3,3-tetramethylbutyl)phenol) [INCI: Bisoctyltriazole], which is available under the trade name Tinosorb® M from CIBA-Chemikalien GmbH.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Advantageous further UV filter substances for the purposes of the present invention are sulfonated, water-soluble UV filters, such as, for example,

phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer;

salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Haarmann & Reimer;

1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethane sulfonic acid) and salts thereof (particularly the corresponding 10-sulfato compounds, in particular the corresponding sodium, potassium or triethanolammonium salts), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)-sulfonic acid and salts thereof.

Advantageous UV filter substances for the purposes of the present invention are also so-called broadband filters, i.e. filter substances which absorb both UV-A and UV-B radiation.

Advantageous broadband filters or UV-B filter substances are, for example, triazine derivatives, such as, for example,

2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA Chemikalien GmbH;

dioctylbutylamidotriazone (INCI: Dioctylbutamidotriazone), which is available under the trade name UVASORB HEB from Sigma 3V;

tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Octyl Triazone), which is sold by BASF Aktiengesellschaft under the trade name UVINUL® T 150.

An advantageous broadband filter for the purposes of the present invention is 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

A further advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The UV filter substances may be oil-soluble or water-soluble.

Advantageous oil-soluble filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone and

UV filters bonded to polymers.

Advantageous water-soluble filter substances are, for example:

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light protection filter substance to be used advantageously according to the invention is ethylhexyl 2-cyano-3,3-diphenylacrylate (octocrylene), which is obtainable from BASF under the name Uvinul® N 539.

The list of specified UV filters which may be used for the purposes of the present invention is not of course intended to be limiting.

Particularly advantageous preparations for the purposes of the present invention which are characterized by high or very high UV-A protection preferably comprise two or more UV-A or broadband filters, in particular dibenzoylmethane derivatives [for example 4-(tert-butyl)-4′-methoxydibenzoylmethane], benzotriazole derivatives [for example 2,2′-methylenebis(6-benzotriazol-2-yl)-4-(1,1,3,3-tetramethylbutyl)phenol)], phenylene-1,4-bis(2-benzimidazyl)-3,3′-5,5′-tetrasulfonic acid or its salts, 1,4-di(2-oxo-10-sulfo-3-bornylidenemethyl)benzene or salts thereof or 2,4-bis{[4-(2-ethylhexyloxy)-2-hydroxy]phenyl}-6-(4-methoxyphenyl)-1,3,5-triazine, in each case individually or in any combinations with one another.

The examples below serve to illustrate the present invention without limiting it. Unless stated otherwise, all amounts, fractions and percentages are based on the weight and the total amount or on the total weight of the preparations.

### EXAMPLE 1 (FOAMY O/W CREAM)

| Emulsion I | % by wt. | % by vol. |
|---|---|---|
| Stearic acid | 3.00 | |
| Cetyl alcohol | 8.50 | |
| PEG-20 stearate | 8.50 | |
| Talc | 2.00 | |
| SiO₂ | 2.00 | |
| Polyacrylic acid | 0.20 | |
| Magnesium aluminum | 0.50 | |
| silicate | | |
| Paraffin oil | 5.00 | |
| Isohexadecane | 2.00 | |
| Glycerol | 5.00 | |
| Sodium hydroxide | q.s. | |
| Preservative | q.s. | |
| Perfume | q.s. | |
| Water, demineralized | ad 100.00 | |
| pH adjusted to | | |
| 6.5-7.5 | | |
| Emulsion I | | 70 |
| Nitrogen | | 30 |

Predispersion of the inorganic gel former and swelling of the hydrocolloid with stirring in the water phase. Combining of the fatty phase heated to 75° C. with the water phase heated to 70° C. Addition of the particulate hydrophobic, hydrophobicized solid-body substances with stirring. Homogenization by means of a toothed-wheel dispersing machine (rotor-stator principle) at 65° C. 45 min stirring with gassing with nitrogen at 0.7 bar and cooling. Addition of the additives at 30° C. (perfume, active ingredients). Homogenization by means of a toothed-wheel dispersing machine (rotor-stator principle) at 27° C.

### EXAMPLE 2 (FOAMY O/W LOTION)

| Emulsion II | % by wt. | % by vol. |
|---|---|---|
| Stearic acid | 2.00 | |
| Myristyl alcohol | 1.50 | |
| Cetylstearyl alcohol | 0.50 | |
| PEG-100 stearate | 3.00 | |
| Talc | 0.05 | |
| Hydroxyethylcellulose | 0.05 | |
| Magnesium aluminum | 0.20 | |
| silicate | | |
| Mineral oil | 5.00 | |
| Hydrogenated | 15.00 | |
| polyisobutene | | |
| Glycerol | 3.00 | |
| Sodium hydroxide | q.s. | |
| Preservative | q.s. | |
| Perfume | q.s. | |
| Water, demineralized | ad 100.00 | |
| pH adjusted to | | |
| 5.0-6.5 | | |
| Emulsion II | | 50 |
| Gas (carbon dioxide) | | 50 |

Predispersion of the inorganic gel former and swelling of the hydrocolloid with stirring in the water phase. Combining of the fatty phase heated to 80° C. with the water phase heated to 72° C. Addition of the particulate hydrophobic, hydrophobicized solid-body substances with stirring. Homogenization by means of a toothed-wheel dispersing machine (rotor-stator principle) at 65° C. 45 min stirring with gassing with carbon dioxide at 1.2 bar and cooling. Addition of the additives at 30° C. (perfume). Homogenization by means of a toothed-wheel dispersing machine (rotor-stator principle) at 30° C.

### EXAMPLE 3 (FOAMY O/W LOTION)

| Emulsion III | % by wt. | % by vol. |
|---|---|---|
| Stearic acid | 5.00 | |
| Cetylstearyl alcohol | 5.50 | |
| PEG-30 stearate | 1.00 | |
| Aluminum starch | 3.00 | |
| octenylsuccinate | | |
| Al₂O₃ | 0.50 | |
| Talc | 0.50 | |
| Polyurethane | 0.10 | |
| Polyacryl methacrylate | 0.10 | |
| Magnesium silicate | 0.10 | |
| Cellulose gum | 0.10 | |
| cyclomethicone | 3.00 | |
| Isoeicosane | 10.00 | |
| Polydecene | 10.00 | |
| Citric acid | 0.10 | |
| Glycerol | 3.00 | |
| Perfume, preservative | q.s. | |
| Sodium hydroxide | q.s. | |
| Dyes etc. | q.s. | |
| Water | ad 100.00 | |
| pH adjusted to | | |
| 6.0-7.5 | | |
| Emulsion III | | 65 |
| Gas (air) | | 35 |

Predispersion of the inorganic gel former and swelling of the hydrocolloids with stirring in the water phase. Combining of the fatty phase heated to 80° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in an open reactor up to 30° C. Addition of the additives at 30° C. (perfume, active ingredients). Homogenization by means of a toothed-wheel dispersing machine (rotor-stator principle) at 25° C.

### EXAMPLE 4 (FOAMY O/W EMULSION MAKE-UP)

| Emulsion IV | % by wt. | % by vol. |
|---|---|---|
| Palmitic acid | 2.00 | |
| Cetyl alcohol | 2.00 | |
| PEG-100 stearate | 2.00 | |
| Polyacrylic acid | 0.10 | |
| Aluminum starch | 0.05 | |
| octenylsuccinate | | |
| Manioc starch | 0.05 | |
| Zeolites | 0.75 | |
| Kaolin | 4.50 | |
| Sodium magnesium | 0.15 | |
| silicate | | |
| Dimethicone | 0.50 | |
| Paraffin oil | 9.50 | |
| Dicaprylyl ether | 2.00 | |
| Glycerol | 3.00 | |
| Mica | 1.00 | |
| Iron oxides | 1.00 | |
| Titanium dioxide | 4.50 | |
| Vitamin A palmitate | 0.10 | |
| Hectorite | 0.10 | |
| Sodium hydroxide | q.s. | |
| Preservative | q.s. | |
| Perfume | q.s. | |
| Water demineralized | ad 100.00 | |
| pH adjusted to | | |
| 6.0-7.5 | | |
| Emulsion IV | | 37 |
| Gas (oxygen) | | 63 |

Predispersion of the inorganic gel formers and swelling of the hydrocolloid with stirring in the water phase. Combining of the fatty and pigment phase heated to 78° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with oxygen at 1.3 bar with cooling to 30° C. Addition of the aluminum starch octenylsuccinate, the manioc starch, the perfume and the active ingredients at 30° C. Homogenization by means of a toothed-wheel dispersing machine (rotor-stator principle) at 25° C.

### EXAMPLE 5 (FOAMY O/W CREAM)

| Emulsion V | % by wt. | % by vol. |
|---|---|---|
| Stearic acid | 4.00 | |
| Cetyl alcohol | 2.00 | |
| PEG-30 stearate | 2.00 | |
| Sorbitan monostearate | 1.50 | |
| Paraffin oil | 5.00 | |
| Cyclomethicone | 1.00 | |
| Vitamin E acetate | 1.00 | |
| Retinyl palmitate | 0.20 | |
| Glycerol | 3.00 | |
| BHT | 0.02 | |
| Na₂H₂EDTA | 0.10 | |
| Polyurethane | 0.10 | |
| Carboxymethylcellulose | 0.05 | |
| Polyacrylic acid | 0.10 | |
| Quaternium-18 hectorite | 0.20 | |
| Magnesium aluminum | 0.10 | |
| silicates | | |
| Silicon dioxide | 0.05 | |
| Talc | 1.00 | |
| Perfume, preservative | q.s. | |
| Dyes | q.s. | |
| Potassium hydroxide | q.s. | |
| Water | ad 100.00 | |
| pH adjusted to | | |
| 5.0-7.0 | | |
| Emulsion V | | 43 |
| Gas (nitrous oxide) | | 57 |

Predispersion of the inorganic gel formers and swelling of the hydrocolloid with stirring in the water phase. Combining of the fatty phase heated to 80° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with nitrous oxide at 0.7 bar with cooling to 30° C. Addition of the additives at 30° C. (perfume, active ingredients). Homogenization by means of a toothed-wheel dispersing machine (rotor-stator principle) at 26° C.

### EXAMPLE 6 (FOAMY O/W LOTION)

| Emulsion VI | % by wt. | % by vol. |
|---|---|---|
| Stearic acid | 4.00 | |
| Cetylstearyl alcohol | 1.00 | |
| PEG-100 stearate | 1.00 | |
| PEG-100 stearate | 1.00 | |
| Distarch phosphate | 0.50 | |
| Paraffin oil | 6.50 | |
| Dimethicone | 0.50 | |
| Vitamin E acetate | 2.00 | |
| Glycerol | 3.00 | |
| Carboxymethylcellulose | 0.05 | |
| Polyacrylic acid | 0.10 | |
| Wheat starch | 0.10 | |
| Magnesium aluminum | 0.50 | |
| silicate | | |
| Kaolin | 0.05 | |
| Talc | 0.50 | |
| Perfume, preservative | q.s. | |
| Dyes etc. | q.s. | |
| Sodium hydroxide | q.s. | |
| Water | ad 100.00 | |
| pH adjusted to | | |
| 6.0-7.5 | | |
| Emulsion VI | | 35 |
| Gas (argon) | | 65 |

Predispersion of the inorganic gel formers and swelling of the hydrocolloid with stirring in the water phase. Combining of the fatty phase heated to 78° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with argon at 1 bar with cooling to 30° C. Addition of the additives at 30° C. (perfume, active ingredients). Homogenization by means of a toothed-wheel dispersing machine (rotor-stator principle) at 23° C.

### EXAMPLE 7 (FOAMY SUNSCREEN CREAM)

| Emulsion VII | % by wt. | % by vol. |
|---|---|---|
| Stearic acid | 1.00 | |
| Cetylstearyl alcohol | 4.00 | |
| Myristyl alcohol | 1.00 | |
| Boron nitride | 1.00 | |
| Kaolin | 0.50 | |
| Silica dimethyl silylate | 1.50 | |
| PEG-20 stearate | 1.00 | |
| Acrylate/C₁₀₋₃₀ alkyl | 0.10 | |
| acrylate crosspolymer | | |
| Hectorite | 0.20 | |
| Quaternium-18 hectorite | 0.10 | |
| Caprylic/capric | 2.00 | |
| triglycerides | | |
| Paraffin oil | 15.50 | |
| Dimethicone | 0.50 | |
| Octyl isostearate | 5.00 | |
| Glycerol | 3.00 | |
| Octyl methoxycinnamate | 4.00 | |
| Butylmethoxydibenzoylmethane | 3.00 | |
| Ethylhexyltriazone | 3.00 | |
| BHT | 0.02 | |
| Na₂H₂EDTA | 0.10 | |
| Perfume, preservative | q.s. | |
| Dyes, etc. | q.s. | |
| Potassium hydroxide | q.s. | |
| Water | ad 100.00 | |
| pH adjusted to 5.0-6.0 | | |
| Emulsion VII | | 35 |
| Gas (helium) | | 65 |

Predispersion of the inorganic gel former (hectorite) and swelling of the hydrocolloids with stirring in the water phase. Predispersion of the quaternium-18 hectorite in the hot fatty phase. Combining of the fatty/photoprotection filter phase heated to 78° C. with the water/photoprotection filter phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with helium at 1 bar with cooling to 30° C. Addition of the additives at 30° C. (perfume). Homogenization by means of a toothed-wheel dispersing machine (rotor-stator principle) at 23° C.

That which is claimed:

1. A self-foaming or foamy cosmetic or dermatological preparation which comprises
a. an emulsifier system comprising:
i. at least one emulsifier A selected from the group consisting of completely neutralized, partially neutralized or unneutralized branched or unbranched, saturated or unsaturated fatty acids with a chain length of from 10 to 40 carbon atoms,
ii. at least one emulsifier B selected from the group consisting of polyethoxylated fatty acid esters with a chain length of from 10 to 40 carbon atoms and with a degree of ethoxylation of from 5 to 100 and
iii. at least one coemulsifier C selected from the group consisting of saturated or unsaturated, branched or unbranched fatty alcohols with a chain length of from 10 to 40 carbon atoms,
b. up to 30% by weight, based on the total weight of the preparation, of a lipid phase,
c. 1 to 90% by volume, based on the total volume of the preparation, of at least one gas selected from the group consisting of air, oxygen, nitrogen, helium, argon, nitrous oxide (N₂O) and carbon dioxide (CO₂),
d. 0.01 to 10% by weight of one or more inorganic thickeners,
e. one or more organic hydrocolloids, and
f. 0.01 to 10% by weight of one or more particulate hydrophobic, hydrophobicized or oil-absorbing solid-body substances.

2. The preparation as claimed in claim 1, wherein the weight ratio of emulsifier A to emulsifier B to coemulsifier C (A:B:C) is a:b:c, wherein a, b and c, independently of one another, are from 1 to 5.

3. The preparation as claimed in claim 1, wherein the weight ratio of emulsifier A to emulsifier B to coemulsifier C (A:B:C) is a:b:c, wherein a, b and c, independently of one another, are from 1 to 3.

4. The preparation as claimed in claim 1, wherein the weight ratio of emulsifier A to emulsifier B to emulsifier C is about 1:1:1.

5. The preparation as claimed in claim 1, wherein the total amount of emulsifier A, emulsifier B and coemulsifier C is from 2 to 20% by weight, based on the total weight of the formulation.

6. The preparation as claimed in claim 1, further comprising at least one additional emulsifier selected from the group consisting of hydrophilic emulsifiers.

7. The preparation as claimed in claim 6, wherein the hydrophilic emulsifier are selected from the group consisting of mono-, di- and trifatty acid esters of sorbitan.

8. The preparation as claimed in claim 6, wherein the total amount of the additional emulsifiers is less than 5% by weight, based on the total weight of the formulation.

9. The preparation as claimed in claim 1, wherein the total amount of hydrocolloids in the cosmetic or dermatological preparation is less than 5% by weight, based on the total weight of the preparation.

10. The preparation as claimed in claim 9, wherein the total amount of hydrocolloids in the cosmetic or dermatological preparation is from 0.1 to 1.0% by weight, based on the total weight of the preparation.

11. The preparation as claimed in claim 1, wherein the volume fraction of the at least one gas is 10 to 80% by volume, based on the total volume of the preparation.

12. The preparation as claimed in claim 1, wherein the at least one gas includes carbon dioxide.

13. The preparation as claimed in claim 1, further comprising one or more substances selected from the group consisting of moisturizers.

14. The preparation as claimed in claim 1, wherein the inorganic thickeners are selected from the group consisting of modified or unmodified, naturally occurring or synthetic sheet silicates.

15. The preparation as claimed in claim 1, wherein the inorganic thickeners are selected from the group consisting of magnesium aluminum silicates, magnesium silicates and sodium magnesium silicates.

16. The preparation as claimed in claim 15, wherein the inorganic thickeners are selected from the group consisting of montmorillonites comprising one or more of bentonites, hectorites and organically modified derivatives thereof.

17. The preparation as claimed in claim 16, wherein the organically modified derivatives include quaternium-18 bentonite, quaternium-18 hectorite, stearalkonium bentonite and stearalkonium hectorite.

18. The preparation as claimed in claim 1, wherein the organic hydrocolloids are selected from the group consisting of:
a) organic, completely synthetic compounds of polyacrylic acids;
b) copolymers and crosspolymers of polyacrylic acid derivatives;
c) ammonium dimethyltauramide/vinylformamide copolymers;
d) copolymers/crosspolymers comprising acryloyldimethyltaurates;
e) hydrophilic gums and hydrophilic derivatives thereof;
f) cellulose or microcrystalline cellulose; and
g) modified carbohydrate derivatives.

19. The preparation as claimed in claim 1, wherein the particulate hydrophobic, hydrophobicized or oil-absorbing solid-body substances are selected from the group consisting of inorganic fillers, inorganic pigments based on metal oxides or other metal compounds which are sparingly soluble or insoluble in water, inorganic pigments based on silicon oxides, silicate derivatives, and microspherical particles based on crosslinked polymethyl methacrylates

20. A method of treating the hair or skin, comprising applying to the hair or skin a self-foaming or foamy cosmetic or dermatological preparation comprising:
a. an emulsifier system comprising:
i. at least one emulsifier A selected from the group consisting of completely neutralized, partially neutralized or unneutralized branched or unbranched, saturated or unsaturated fatty acids with a chain length of from 10 to 40 carbon atoms,
ii. at least one emulsifier B selected from the group consisting of polyethoxylated fatty acid esters with a chain length of from 10 to 40 carbon atoms and with a degree of ethoxylation of from 5 to 100 and
iii. at least one coemulsifier C selected from the group consisting of saturated or unsaturated, branched or unbranched fatty alcohols with a chain length of from 10 to 40 carbon atoms,
b. up to 30% by weight, based on the total weight of the preparation, of a lipid phase,
c. 1 to 90% by volume, based on the total volume of the preparation, of at least one gas selected from the group consisting of air, oxygen, nitrogen, helium, argon, nitrous oxide (N₂O) and carbon dioxide (CO₂),
d. 0.01 to 10% by weight of one or more inorganic thickeners,
e. one or more organic hydrocolloids, and
f. 0.01 to 10% by weight of one or more particulate hydrophobic, hydrophobicized or oil-absorbing solid-body substances.
